(19) United States
(12) Patent Application Publication
(10) Pub. No.: US 2002/0166147 A1
(43) Pub. Date: Nov. 7, 2002

(54) Method of increasing crop yield

(75) Inventors: Anthony Jabar JR., Waterville, ME (US); G. Noel Currie III, Presque Isle, ME (US)

Correspondence Address: NIXON PEABODY LLP, 101 Federal Street, Boston, MA, 02110, US

(73) Assignee: Global Protein Products

(21) Appl. No.: 10/056,803

(22) Filed: Jan. 24, 2002

Related U.S. Application Data
(60) Provisional application No. 60/263,781, filed on Jan. 24, 2001.

(51) Int. Cl. A01N 43/02; A01H 5/00

(52) U.S. Cl. 800/298; 504/140

(57) ABSTRACT

Plants, soils, seeds or seed pieces of agricultural or horticultural value are treated before, during, or after planting with a peptide-polysaccharide complex. This composition, a solution or dry flowable powder peptide-polysaccharide complex, is used as a plant, soil, seed or seed piece treatment for commercial crops to enhance germination, emergence, root mass development, plant growth crop maturity and ultimately increase crop yield. Methods of preparation and methods of use of the peptide-polysaccharide complex are provided.

### FIELD OF THE INVENTION

The present invention relates to methods and compositions used to promote seed germination, emergence, healthy root development, plant growth, disease resistance, maturity and ultimately increase crop yield in agricultural commodities. The present invention also relates to crop seeds and crop seed pieces treated with the compositions and methods of the present invention.

### TECHNICAL BACKGROUND

Economic demands, environmental concerns, and ecological considerations require that farmers continually improve their agricultural practices. Seed today is more expensive, land more valuable, and regulatory compliance more stringent than any other time in history. These economic demands require that farmers utilize the most cost efficient practices in order to generate the highest crop yields. Complicating this economic balance is the increasing consumer demand to utilize fewer chemicals and with lower toxicity. Additionally, environmental considerations such as contaminated runoff, spray drift, and worker safety continue to add pressure to the agricultural practices balance. Finally, ecological considerations have led to integrated pest management systems which further challenge the farmer's ability to produce crop yields and quality within the economic constraints prevalent in today's market.

Plant, soil and seed treatments are used on almost every commercial crop on the market today. In this era of intensified agriculture, the seed is often modified and exclusively owned and thus it is more important than ever to decrease the risk of plant and seed disease once expensive seed has been purchased. It is well known that protecting seed during the early part of the planting season is vital to obtaining high crop yields. Plant protection during the growing season is also of vital importance in obtaining high quality, high yielding crops.

The plants and seeds are subjected to a wide variety of conditions, which can negatively impact germination, emergence, root mass development, nutrient uptake, plant growth, and ultimately crop yield. Pathogenic fungi present a major challenge for growers. Seed storage and seed planting provide fungi with a favorable climate in which to propagate, leading to seed damage, plant damage, and crop yield losses. Unfortunately there are many genera of pathogenic fungi with which the grower must deal. Owing to the variety of fungi, there is no universal antifungal compound, which is effective on all types. Additionally, resistant type fungi development contributes to lower overall effectiveness of antifungal compounds, synthetic or otherwise. Ascochyta, Botrytis, Erypsiphe, Fusarium, Gaeumanomyces, Pythium, Rhizoctonia, Sclerotinia, and Verticillium are just a few of the many genera of fungi with which growers must contend.

In addition to disease control a seed treatment must also provide for optimum germination, emergence, and root development. Absorption of macro and micronutrients is essential for high crop yield development. All of this must be accomplished even under adverse growing conditions. Such a seed treatment is highly desirable.

A new class of peptide-polysaccharides has been recently introduced. McArdle in U.S. Pat. Nos. 5,942,123, 5,747,416, 5,645,880, discloses these peptide-polysaccharides for a number of uses. However, there has been no recognition of their use as plant, soil or seed treatment compositions to promote germination, emergence, root mass, plant growth and higher crop yields.

### SUMMARY OF THE INVENTION

It is therefore an object of this invention to provide a plant, soil and seed treatment composition which enhances rapid germination, uniform and early emergence, healthy root mass, higher rates of photosynthesis, plant protection, early maturity and ultimately increase crop yield.

It is also an object of this invention to provide a plant, soil and seed treatment composition which is nontoxic and biodegradable, which will have minimal impact on worker safety, the environment, and the ecological considerations of the farm.

It is also an object of this invention to provide a composition, which is easily manufactured and easily applied using conventional and commercially available application equipment.

It is also an object of this invention to provide a coated crop seed or seed piece which exhibits increased germination, uniform and early emergence, healthy root mass development, disease resistance, and increased crop yield.

In one embodiment, the present invention provides a method of increasing crop yield. In another embodiment, the present invention provides a method for accelerating crop emergence. In a further embodiment, the present invention provides a method for accelerating crop maturity. In these methods an effective amount of a composition comprising a peptide and a polysaccharide is administered on a seed of the crop or to a soil in which the crop is cultivated. In the composition, the peptide is present in an amount of 2-90% by weight of the dry weight of the total peptide-polysaccharide complex and the polysaccharide is present in an amount of 10-98% by weight of the dry weight of the peptide-polysaccharide complex. In a preferred embodiment, the peptide is present in an amount of 2-30% by weight of the dry weight of the peptide-polysaccharide complex and the polysaccharide is present in an amount of 70%-98% by weight of the dry weight of the peptide-polysaccharide complex. The administration may be performed before seeding of said crop, simultaneously with seeding of the crop or after seeding of the crop.

In another embodiment, the invention provides a seed composition comprising a crop seed and coating comprising a peptide and a polysaccharide, wherein the peptide is present in an amount of 2-90% by weight of the dry weight of the total peptide-polysaccharide complex and the polysaccharide is present in an amount of 10-98% by weight of the dry weight of the peptide-polysaccharide complex. In a preferred embodiment, the peptide is present in an amount of 2-30% by weight of the dry weight of the peptide-polysaccharide complex and the polysaccharide is present in an amount of 70%-98% by weight of the dry weight of the peptide-polysaccharide complex. In one embodiment, the crop seed is a potato or a grain. In a preferred embodiment, the grain is barley.

The peptide for use in the composition can be isolated from a natural or recombinant source. Preferably, the natural source is of plant origin including maize, rice, tobacco, alfalfa, wheat, barley, soybean, and peanuts. A preferred plant peptide is zein. Alternatively, the peptide can be isolated from animal sources including milk, eggs, animal epidermal tissue, or animal connective tissue.

The polysaccharide for use in the composition can be isolated from a natural or recombinant source. Preferably the polysaccharide is a cellulosic derivative. Preferred cellulosic derivatives include carboxymethylcellulose, methylcellulose, hydroxypropyl cellulose, hydroxypropyl methylcellulose, and microcrystalline cellulose.

In another embodiment, the polysaccharide is starch or a starch derivative. Starch or starch derivatives include, for example, tapioca starch, potato starch, rice starch, wheat starch. Modified versions may also be used and include, for example, pregelatinized starch, oxidized starch, ethylated starch, starch dextrins, or maltodextrin.

In a further embodiment, the polysaccharide is pectin. The polysaccharide may be from seaweed and can include, for example, agar, alginate, carrageenan and fucellaran.

In another embodiment, the polysaccharide is derived from an exudate gum polysaccharide and, can include, for example, gum arabic, gum ghatti, gum karaya, and gum tragacanth.

The polysaccharide may be derived from seed gum such as guar gum or locust bean gum. Alternatively, the polysaccharide is derived from microbial fermentation and includes, for example, xanthan gum or gellan gum. Guar gum may be used. In the preferred embodiment, the polysaccharide is guar gum, gum arabic, carrageenan, alginates, or any of the celluloses.

Preferred polysaccharides include the celluloses, guar gum, gum arabic, carrageenan, and alginates.

Additional features and advantages of the invention will be set forth in the detailed description which follows, and in part will be readily apparent to those skilled in the art from that description or recognized by practicing the invention as described herein, including the detailed description which follows, the claims, as well as the appended drawings.

It is to be understood that both the foregoing general description and the following detailed description are merely exemplary of the invention, and are intended to provide an overview or framework for understanding the nature and character of the invention as it is claimed.

### DESCRIPTION OF THE PREFERRED EMBODIMENTS OF THE INVENTION

The present invention provides methods of increasing crop yield and accelerating crop emergence and maturity. In these methods an effective amount of a composition comprising a peptide and a polysaccharide is administered on a seed of the crop or to a soil in which the crop is cultivated. In the composition, the peptide is present in an amount of 2-90% by weight of the dry weight of the total peptide-polysaccharide complex and the polysaccharide is present in an amount of 10-98% by weight of the dry weight of the peptide-polysaccharide complex. In a preferred embodiment, the peptide is present in an amount of 2-30% by weight of the dry weight of the peptide-polysaccharide complex and the polysaccharide is present in an amount of 70%-98% by weight of the dry weight of the peptide-polysaccharide complex. The administration may be performed before seeding of said crop, simultaneously with seeding of the crop or after seeding of the crop.

The composition for use in the present invention includes at least one peptide and at least one polysaccharide. In the composition, the peptide is present in an amount of 2-90% by weight of the dry weight of the total peptide-polysaccharide complex and the polysaccharide is present in an amount of 10-98% by weight of the dry weight of the peptide-polysaccharide complex. In a preferred embodiment, the peptide is present in an amount of 2-30% by weight of the dry weight of the peptide-polysaccharide complex and the polysaccharide is present in an amount of 70%-98% by weight of the dry weight of the peptide-polysaccharide complex. The peptide can be obtained from any natural, transgenic, or synthetic source. Natural sources include peptides produced by naturally occurring organisms or hybrids of naturally occurring organisms. Transgenic sources include but are not limited to organisms produced through genetic engineering. Synthetic sources include peptides produced by artificial chemical synthesis.

The peptide sources include but are not limited to maize, rice, tobacco, alfalfa, wheat, barley, soybean, peanuts, milk, eggs, animal epidermal tissue, animal connective tissue, etc. Peptides of plant origin are preferred and include, but are not limited to, corn gluten, corn zein, wheat gliadin, wheat glutenin, wheat gluten, barley hordein, soy protein, soy protein isolates, peanut protein. In certain embodiments, peptides of animal origin can be used and include, but are not limited to, keratin, collagen, gelatin, whey protein, casein, and egg albumin. In a preferred embodiment of the present invention the peptides are zein, corn gluten, gliadin, glutenin, wheat gluten, barley hordein, soy protein, whey protein, casein, or mixtures thereof.

The polysaccharides utilized in the present invention are, in particular, hydrocolloid polysaccharides derived from plant, animal or microbial sources. Polysaccharides useful in the present invention include, but are not limited to, cellulosic derivatives such as carboxymethylcellulose, methylcellulose, hydroxypropyl cellulose, hydroxypropyl methylcellulose, microcrystalline cellulose, etc., starches and derivatives including, but not limited to, corn starch, tapioca starch, potato starch, rice starch, wheat starch, and modified versions thereof such as pregelatinized starch, oxidized starch, ethylated starch, starch dextrins, maltodextrin, etc. Additional polysaccharides useful in the present invention include, but are not limited to, pectin, polysaccharides derived from seaweed such as agar, alginates, carrageenan, and fucellaran, exudate gum polysaccharides such as gum arabic, gum ghatti, gum karaya, and gum tragacanth, seed gums such as guar gum and locust bean gum, polysaccharides derived from microbial fermentation such as xanthan gum and gellan gum, and nitrogen containing polysaccharides such as chitosan. Polysaccharides of the type described herein produced by transgenic organisms can also be useful in the present invention. In a preferred embodiment of this invention, the polysaccharides are guar gum, starch dextrins, pectin, gum arabic, and mixtures thereof.

The total peptide-polysaccharide composition is defined as the total weight of the peptide and the polysaccharide. The peptide is present in an amount between about 2% and 90% by weight of the peptide-polysaccharide composition, preferably in the amount ranging between about 5% and 30%.

The polysaccharide is present in an amount between 10% and 98% by weight of the total peptide-polysaccharide composition, preferably in the amount ranging from 70% to 95%. The total peptide-polysaccharide composition is defined as the total weight of the peptide and the polysaccharide.

To prepare the composition, the peptide is first dissolved or dispersed in an appropriate solvent. The solvent can be an aqueous system or an organic solvent based system depending on the solubility parameters of the peptide. In the preferred embodiment of this invention the peptide, zein, is dissolved in a water/alcohol solution containing a ratio of water to alcohol of 30/70 by volume to form a solution of peptide in aqueous alcohol. The polysaccharide is subsequently added to the peptide aqueous alcohol solution thereby forming a peptide-polysaccharide complex in aqueous alcohol. The peptide-polysaccharide complex solution can then be dried to a free flowing powder using conventional drying or evaporation equipment; however it may be preferable to maintain the peptide-polysaccharide complex in solution for certain applications.

In another embodiment, to prepare the composition, a solution of peptide is dissolved in an appropriate solvent, which is capable of dissolving the peptide. The solution is filtered to remove the non-peptide material. The polysaccharide is added to the filtered peptide solution. The peptide-polysaccharide solution is then dried by conventional methods with care not to exceed 140° C. Conventional drum drying, spray drying, or oven drying is easily accomplished utilizing commercially available equipment. The dried powder is then ground to an appropriate size making it thereby re-dispersible in solution.

In an alternative embodiment, the composition can be prepared by blending a dry peptide with a dry polysaccharide. This dry blending can be carried out in conventional dry blending equipment.

The peptide-polysaccharide dry flowable powder compositions made according to the methods above are then applied to the plant, soil, seed or seed piece by conventional application equipment. Hopper blending, auger mixing, barrel dusting, disking, and spray dusting are all commercially available methods and are capable of treating plant, seed or seed pieces with the peptide-polysaccharide composition. Additionally, the peptide-polysaccharide compositions may be added directly to the soil by broadcast methods, disking, or row application.

A preferred effective amount of the composition is between 0.5-2.0 lbs./acre.

Alternatively, the peptide-polysaccharide can be re-dispersed in water or other appropriate solvent, with or without the aid of conventional dispersants, emulsifiers or solubilizing agents, and applied to the plant, soil, seed or seed piece as a solution. A bath type application can be utilized in the present invention followed by drying. Spray equipment may also be used to apply the peptide-polysaccharide solution, again followed by drying. Drying can be accomplished by forced air circulation, heated air circulation, atmospheric air drying, oven drying, or any other means which will have the effect of removing excess moisture from the plant, seed or seed piece. Side dressing, drip application and foliar applications to the plants do not require any artificial drying methods.

Advantageously, additives may be added to the aqueous alcohol solution to promote stability of the peptide-polysaccharide complex. Additives include, but are not limited to, solubilizing agents such as glycol, propylene glycol, or other low molecular weight alcohols; surfactants such as alkylpolyglucosides, fatty alcohols, fatty acids, or alkylbenzenesulfonates and dispersants; emulsifiers such as lecithin or sorbitan monooleate; pH control agents such as mineral acids and their salts, organic acids and their salts, bases, both organic and inorganic; buffers such as phosphates, acetates and carbonates; anti-microbial compounds such as BHT, methyl or propylparaben, benzoic acid, sorbic acid, propionic acid and their salts; chelating agents such as EDTA, MEA or TEA; and other such additives generally known or apparent to those skilled in the art.

The composition can be effectively used to treat soils, plant seeds or seed pieces either by direct application to the seed or seed piece before planting or, alternatively, the composition may be added during the planting process along with the seeds or seed pieces.

The present compositions can also be added pre or post planting as either a foliar application, as a side dressing, or as a soil amendment admixed with the soil using conventional agricultural practices such as broadcasting, disking or row application.

The peptide-polysaccharide compositions can be admixed with inert agents, bulking agents, or diluent materials in order to uniformly distribute the peptide-polysaccharide onto the plant, soil, and seed or seed piece surface. Materials which can be used in the present invention include, but are not limited to clay, talc, limestone, quick lime, silica, hydrated silica, bentonite, salts of organic acids, organic acids, surfactants, dispersants, emulsifiers, solvents, ash, composted materials, tree bark, and mixtures thereof. When admixing these materials care should be taken to insure that the appropriate amount of peptide-polysaccharide is distributed uniformly onto the plant, soil, seed or seed piece.

Similarly, other functional ingredients may be added to the peptide-polysaccharide complex to simplify distribution of these ingredients onto the plant, soil, seed, or seed piece. When admixing these materials care should be taken to insure that the appropriate amount of peptide-polysaccharide is distributed uniformly onto the plant, soil, seed or seed piece.

The present invention can be used to enhance the germination, emergence, root mass development, disease resistance, photosynthetic rate, plant growth, and crop yield of a variety of agricultural commodities including but not limited to: vegetables, such as asparagus, beans, beets, broccoli, brussels sprouts, cabbage, carrots, cauliflower, celery, chayote, corn, cucumbers, eggplant, kohlrabi, okra, onions, garlic, parsnips, peas, peppers, potatoes, pumpkins, radishes, rutabagas, squash, turnips, lettuce, kale, collards, spinach, sweet potato, sugar beets, etc.; fruits, such as apples, apricots, avocados, bananas, cherries, coconuts, dates, grapes, guava, lychee, mangoes, melons, nectarines, papaya, peaches, pears, persimmons, pineapples, plantains, plums, pomegranates, prunes, stone fruit, strawberries, tomatoes, blueberries, raspberries, blackberries, and citrus fruits, such as grapefruit, oranges, lemons, limes, clementines and tangerines.

### EXAMPLE 1

Potato Seed Treatment—Aroostook County, Maine

To prepare the composition, zein (Freeman Industries, Tuckahoe, N.Y.) was dissolved in a water/alcohol solution containing a ratio of water to alcohol of 30/70 by volume to form a solution of zein in aqueous alcohol. The guar gum (Monson Chemical, Leominster, Mass.) was subsequently added to the peptide aqueous alcohol solution thereby forming a peptide-polysaccharide complex in aqueous alcohol. The peptide-polysaccharide complex was then dried to a free flowing powder using conventional air flow drying. A peptide-polysaccharide complex composed of 8% zein/ 92% guar gum dry flowable powder mixed with calcium carbonate was applied to cut potato seed pieces to determine the effect on emergence, seed piece integrity, total yield, and marketable yield. The peptide-polysaccharide complex was applied at three different rates of addition, 500 PPM, 750 PPM, and 1000 PPM. The following data summarizes the results.

| PPM | | % Seed Integrity | Total | |
|---|---|---|---|---|
| (15 DAP) | % Emergence | # Tubers | Yield | % Marketable |
| 0 | 48 | 20-40 | 35 | 80 |
| 500 | 48 | 80-100 | 38 | 85 |
| 750 | 53 | 80-100 | 55 | 88 |
| 1000 | 63 | 80-100 | 45 | 82 |

DAP= Days after planting

The protection offered by the composition, at the critical early stage of plant development when the seed piece is most vulnerable, is evidenced by the increased rate of emergence and seed piece integrity. The application of the peptide-polysaccharide complex resulted in increased marketable yield, as measured by the number of tubers of a size equal to 3 inches in length or greater. This results in a yield increase ranging from 15-73% of economically valuable tubers.

### EXAMPLE 2

Grain Seed Treatment—Aroostook County, Maine

A plot of cereal grains, barley, was established using a peptide-polysaccharide complex (same as in Example 1) at a rate of 80 PPM. The stand was evaluated for height and compared to an untreated control stand.

Results

| | Pre Harvest height (inches) | % Increase |
|---|---|---|
| Control (No treatment) | 36 | 0 |
| 80 PPM | 42 | 16.7 |

### EXAMPLE 3

Potato Seed Treatment—Idaho Falls, Id.

100 acres of potatoes were planted with a peptide-polysaccharide complex. To prepare the composition, zein (Freeman Industries, Tuckahoe, N.Y.) was dissolved in a water/alcohol solution containing a ratio of water to alcohol of 30/70 by volume to form a solution of zein in aqueous alcohol. Guar gum (Monson Chemical, Leominster, Mass.) was subsequently added to the peptide aqueous alcohol solution thereby forming a peptide-polysaccharide complex in aqueous alcohol. The peptide-polysaccharide complex was then dried to a free flowing powder using conventional air flow drying. A peptide-polysaccharide complex composed of 8% zein/92% guar gum at a rate of 2 lbs of peptide-polysaccharide per 2000 lbs of seed potato. The peptide-polysaccharide was mixed with calcium carbonate. The peptide-polysaccharide complex was evaluated versus a conventional fungicide treatment (TOPS-MZ, Gustafson, Plano Tex.) at the same rate of addition.

Results - Yield

| Yield | | | Yield per | | #Tubers | |
|---|---|---|---|---|---|---|
| 10 ft row (lbs.) | | % Inc. | Plant (lbs) | % Inc. | 10 ft row | % Inc. |
| C | 20.04 | 0 | 2.23 | 0 | 66.5 | 0 |
| T | 24.41 | 21.8 | 2.87 | 28.7 | 75.5 | 13.5 |

C = control
T = treated

These results are demonstrative for a large field plot evaluation of the peptide-polysaccharide complex. A 21.8% increase in yield resulted in the production of an additional 650,000 pounds of potatoes for this 100 acre plot.

The preceding examples are to be evaluated as illustrative and are not intended to limit the scope of this invention.

It is also to be understood that the following claims are intended to cover all of the generic and specific features of the invention herein described and all statements of the scope of the invention which, as a matter of language, might be said to fall there between. Particularly, it is to be understood that in said claims, ingredients or compounds recited in the singular are intended to include compatible mixtures of such ingredients wherever sense permits.

We claim:

1. A method of increasing crop yield comprising administering an effective amount of a composition comprising a peptide and a polysaccharide on a seed of said crop or to a soil in which said crop is cultivated, wherein the peptide is present in an amount of 2-90% by weight of the dry weight of the total peptide-polysaccharide complex and the polysaccharide is present in an amount of 10-98% by weight of the dry weight of the peptide-polysaccharide complex.

2. A method for accelerating crop emergence comprising administering an effective amount of a composition comprising a peptide and a polysaccharide on a seed of said crop or to a soil in which said crop is cultivated, wherein the peptide is present in an amount of 2-90% by weight of the dry weight of the total peptide-polysaccharide complex and the polysaccharide is present in an amount of 10-98% by weight of the dry weight of the peptide-polysaccharide complex.

3. A method accelerating crop maturity comprising administering an effective amount of a composition comprising a peptide and a polysaccharide on a seed of said crop or to a soil in which said crop is cultivated, wherein the peptide is present in an amount of 2-90% by weight of the dry weight of the total peptide-polysaccharide complex and the polysaccharide is present in an amount of 10-98% by weight of the dry weight of the peptide-polysaccharide complex.

4. The method of claims 1, 2 and 3, wherein the peptide is present in an amount of 2-30% by weight of the dry weight of the peptide-polysaccharide complex and the polysaccharide is present in an amount of 70%-98% by weight of the dry weight of the peptide-polysaccharide complex.

5. The method of claims 1, 2 and 3, wherein administering is performed before seeding of said crop.

6. The method of claims 1, 2 and 3, wherein administering is performed simultaneously with seeding of said crop.

7. The method of claim 1, wherein administering is performed after seeding of said crop.

8. A seed composition comprising a crop seed and coating, said coating comprising a peptide and a polysaccharide, wherein the peptide is present in an amount of 2-90% by weight of the dry weight of the total peptide-polysaccharide complex and the polysaccharide is present in an amount of 10-98% by weight of the dry weight of the peptide-polysaccharide complex.

9. The composition of claim 8, wherein the peptide is present in an amount of 2-30% by weight of the dry weight of the peptide-polysaccharide complex and the polysaccharide is present in an amount of 70%-98% by weight of the dry weight of the peptide-polysaccharide complex.

10. The composition of claim 8, wherein the crop seed is a potato.

11. The composition of claim 8, wherein the crop seed is a grain.

12. The composition of claim 11, wherein the grain is barley.

13. The method of claims 1, 2 and 3, wherein the peptide is isolated from a natural, synthetic or recombinant source.

14. The method of claims 1, 2 and 3, wherein the peptide is zein.

15. The method of claims 1, 2 and 3, wherein the polysaccharide is isolated from a natural, synthetic or recombinant source.

16. The method of claim 15, wherein the polysaccharide is a cellulosic derivative.

17. The method of claim 16, wherein the cellulosic derivative is selected from a group consisting of carboxymethylcellulose, methylcellulose, hydroxypropyl cellulose, hydroxypropyl methylcellulose, and microcrystalline cellulose.

18. The method of claims 1, 2 and 3, wherein the polysaccharide is starch or a starch derivative.

19. The method of claims 1, 2 and 3, wherein the polysaccharide is pectin.

20. The method of claims 1, 2 and 3, wherein the polysaccharide is derived from an exudate gum polysaccharide.
